(19) United States
(12) Patent Application Publication
(10) Pub. No.: US 2026/0248421 A1
(43) Pub. Date: Aug. 27, 2026

(54) APTAMER BIOSENSORS WITH MIXED-CHARGE MONOLAYER ANTI-FOULING CAPABILITY

(71) Applicant: University of Cincinnati, Cincinnati, OH (US)

(72) Inventors: Jason Heikenfeld, Cincinnati, OH (US); Thomas Young, Cincinnati, OH (US)

(21) Appl. No.: 19/163,080

(22) PCT Filed: Mar. 8, 2024
(86) PCT No.: PCT/US2024/019279

§ 371 Date: Sep. 8, 2025

Related U.S. Application Data
(60) Provisional application No. 63/451,011, filed on Mar. 9, 2023.

(51) Int. Cl. A61B 5/1473; A61B 5/145

(52) U.S. Cl. CPC A61B 5/14735; A61B 5/1451

(57) ABSTRACT

A device for continually sensing at least one analyte in a sample fluid via measurement of the analyte is provided. The device has a sensor with a surface having a plurality of aptamers that bind to the analyte. The plurality of aptamers carries at least one tag. The surface also has a protective layer that protects the surface from fouling in between the aptamers. The protective layer has a monolayer of molecules that form a boundary with the sample fluid. Further, the monolayer of molecules is a mixed charge monolayer.

### CROSS REFERENCE TO RELATED APPLICATIONS

This application claims priority to U.S. Provisional Application Ser. No. 63/451,011, filed Mar. 9, 2023, which application is hereby incorporated by reference in its entirety.

### FIELD OF THE INVENTION

This invention relates generally to placement of biosensors into the skin with multiple depths of penetration accessible by a single device.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Electrochemical aptamer sensors can identify the presence and/or concentration of an analyte of interest via the use of an aptamer sequence that specifically binds to the analyte of interest. These sensors include aptamers attached to an electrode, wherein each of the aptamers has a redox active molecule (redox tag) attached thereto. The redox couple can transfer electrical charge to or from the electrode. When an analyte binds to the aptamer, the aptamer changes shape, bringing the redox couple closer to or further from, on average, the electrode. This results in a measurable change in electrical current that can be translated to a measure of presence or concentration of the analyte. When used in this manner, then, aptamers are an example of an affinity-based biosensor.

A major unresolved challenge for aptamer sensors and other affinity-based biosensors (particularly those where the aptamers are bonded to the working electrode) is the lifetime of the sensors, especially for applications where continuous operation is required (“continuous” referring to multiple measurements over time by the same device). Such aptamer sensors are susceptible to degradation due to, among other things, desorption of the aptamers themselves from the electrode, and/or desorption of the protective layer molecules (such as mercaptohexanol) from the electrode. The aptamers and the protective molecules together form a monolayer which can be referred to as a sensing monolayer. The protective layer portion of the sensing monolayer (1) ensures that the aptamer conformation change when binding to an analyte is not physically hindered by foulants, and (2) reduces electrical background current (including oxygen reduction current), which would otherwise wash-out the measured signal from the aptamer and redox tag.

Current methods of fabrication of these devices uses a very simple and convenient approach of forming a partial monolayer of aptamer by thiol bonding to a gold electrode via incubation of the electrode in solution including aptamer(s), followed by forming a more complete monolayer including the protective molecule such as mercaptohexanol (via incubation of the electrode in mercaptohexanol solution). This process is quite fortuitous for researchers because not only does a monolayer of mercaptohexanol reduce background current, but mercaptohexanol monolayers as-typically-formed have at least one feature such as defects, for example, which allow for electron transfer between the redox tag and the electrode, these defects being few and/or small enough to minimize oxygen reduction current and other major sources of background current. Furthermore, mercaptohexanol monolayers have adequate defects for electron transfer to support a zero-gain frequency that allows two frequency or comparable self-calibration techniques. Lastly, mercaptohexanol has enough surface fouling resistance to allow for short-term in-lab experiments in biofluids such as blood or serum.

Therefore, researchers have had at their disposal a very ‘convenient’ way to make aptamer sensors for research applications. However, most aptamer researchers have not historically been motivated to address longevity of aptamer sensors, and the same monolayer approach that is so convenient is also inherently fragile as the monolayer is able to desorb over time. Part of this cause for desorption is that each portion of the monolayer is a single molecule that has a single bond to the electrode, and statistically or energetically breaking one of these bonds with the electrode is not that difficult with conventional monolayer chemistries, especially at elevated temperatures such as body temperature. Multiple bonds to the gold could alleviate this challenge, but also may lack the tight packing density required for a low background current during measurement. According to leading experts in the 2022 review article, see Shaver, et al., “The challenge of long-term stability for nucleic acid-based electrochemical sensors,” Current Opinion in Electrochemistry (2022), 32:100902 (https://doi.org/10.1016/j.coelec.2021.100902), “Unfortunately, these chemistries desorb over time when exposed to environmental or experimental factors like, for example, dry air, high temperature, voltage pulsing, and biological fluids. This desorption process simultaneously removes sensing moieties and passivating thiols from the electrode surface, prohibiting their deployment for more than a few hours.” Clearly, even to the experts in the field, aptamer sensor longevity had remained an unresolved problem with no obvious solutions for achieving sensor longevity for multiple days or weeks. Even as alternate methods are developed for extending longevity of protective layers, these methods must also support proper electron-transfer for sensor signaling, ideally allow use of one or more calibration-free methods of operation, and prevent over-fouling that otherwise would inhibit movement of the aptamer and therefore proper signaling of the sensor. Inventors Young and co-inventor Heikenfeld recently published a breakthrough on aptamer sensor longevity by stabilizing an alkythiolate based protective layer system for aptamer sensors with greater than one week of operational longevity, see Watkins, et al., “Week-Long Operation of Electrochemical Aptamer Sensors: New Insights into Self-Assembled Monolayer Degradation Mechanisms and Solutions for Stability in Serum at Body Temperature”, ACS Sens. 2023, 8, 3, 1119-1131, Publication Date: Mar. 8, 2023, https://doi.org/10.1021/acssensors.2c02403 (herein called the ‘Young and Heikenfeld longevity paper’). The paper describes in detail degradation mechanisms for aptamer sensors and shows the utility of membrane protection to preserve aptamer sensor functionality by preventing fouling of the monolayer over multiple days.

For sensing larger analytes (>1's or >10's kDa) or for multiweek sensing, membrane protection may not be adequate because if the target analyte is to reach the sensing surface, then also some foulants may still reach the sensor surface. Motivated to resolve aptamer sensor operation without membrane protection, even if just for short term (<24 hour) sensor operation, researchers have implemented a host of approaches to mitigate the effects of sensor degration in whole biofluids, for example including two frequency ‘calibration free’ operation as taught in Li H, Dauphin-Ducharme P, Ortega G, Plaxco KW. Calibration-Free Electrochemical Biosensors Supporting Accurate Molecular Measurements Directly in Undiluted Whole Blood. J Am Chem Soc. 2017 August 16; 139(32): 11207-11213. doi: 10.1021/jacs.7b05412. Epub 2017 August 2. PMID: 28712286; PMCID: PMC 6519131, and for example including ‘kinetic differential measurement’ as taught in Arroyo-Currás N, Somerson J, Vieira PA, Ploense K L, Kippin T E, Plaxco K W. Real-time measurement of small molecules directly in awake, ambulatory animals. Proc Natl Acad Sci USA. 2017 Jan. 24; 114(4): 645-650. doi: 10.1073/pnas.1613458114. Epub 2017 Jan. 9. PMID: 28069939; PMCID: PMC 5278471.

The Young and Heikenfeld longevity paper referenced above, was able to demonstrate for the first time that fouling was the remaining challenge for aptamer biosensors achieving multiday or even multiweek operation, with their paper subsection titled ‘Fouling Affects Electron Transfer Rates and Has a Significant Impact on Freedom of Mobility of The Aptamer’. As demonstrated in the Young and Heikenfeld longevity paper, and in a previous paper Li H, Dauphin-Ducharme P, Arroyo-Currás N, Tran C H, Vieira P A, Li S, Shin C, Somerson J, Kippin TE, Plaxco K W. A Biomimetic Phosphatidylcholine-Terminated Monolayer Greatly Improves the In Vivo Performance of Electrochemical Aptamer-Based Sensors. Angew Chem Int Ed Engl. 2017 Jun. 19; 56(26): 7492-7495. doi: 10.1002/anie.201700748. Epub 2017 March 28. PMID: 28371090; PMCID: PMC 5660315, a zwitterionic terminated monolayer can be utilized to reduce fouling of the sensor surface and reduce or remove the need for a protective membrane. However, zwitterionic terminated monolayers can also bring disadvantages to sensor operation, and alternative antifouling approaches may still be required.

Therefore, a need still exists for alternative antifouling chemistries that enable optimal sensor operation in terms of maintaining accuracy and in terms of achieving the desired detection ranges for periods of multiple days or even multiple weeks.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with biofluid and analytes.

One aspect of the present invention is directed to a device for continually sensing at least one analyte in a sample fluid via measurement of the analyte. The device includes at least one sensor. The sensor's surface has a plurality of aptamers that bind to the analyte. The aptamers carry at least one tag that changes in at least one parameter as analyte binds to the aptamers. The sensor's surface also has a protective layer that protects the surface from fouling in between the aptamers. The protective layer further comprises a monolayer of molecules that form a boundary with the sample fluid. The monolayer of molecules is a mixed charge monolayer of at least a first molecule and a second molecule. The first molecule has a first charge that imparts a net negative charge at the boundary with the sample fluid. The second molecule has a second charge that imparts a net positive charge near the boundary with the sample fluid. Compared to other antifouling surface chemistries, this novel approach for aptamer sensors can improve electron transfer rates, improve overall magnitude of electron transfer current, and can create an optimal binding affinity for the analyte to the aptamer and resulting range of detection for analyte.

The present invention involves a device for continually sensing at least one analyte in a sample fluid via measurement of the analyte. The device includes at least one sensor having a surface. The surface has a plurality of aptamers that bind to the analyte. The plurality of aptamers carries at least one tag that changes in at least one parameter as analyte binds to the aptamers. The surface also has a protective layer that protects the surface from fouling in between the aptamers. The protective layer has a monolayer of molecules that form a boundary with the sample fluid. Further, the monolayer of molecules is a mixed charge monolayer of at least a first molecule and a second molecule. The first molecule has a first charge that imparts a net negative charge at the boundary with the sample fluid. The second molecule has a second charge that imparts a net positive charge near the boundary with the sample fluid.

In one embodiment, the first charge and the second charge have a magnitude of at least one electron charge. In another embodiment, the monolayer of molecules comprises a repeating hydrophobic chain of molecules that is selected from the group consisting of two or more methyl groups, two or more phenyl groups, and combinations thereof. In one embodiment, the mixed charge monolayer comprises one or more subsets of molecules, each subset of molecules having a terminus, and further, the mixed charge monolayer has a mixed charge at the terminus of at least a subset of the molecules. In another embodiment, the mixed charge monolayer has a mixed charge located along an interior chain of at least a subset of the molecules. In one embodiment, the mixed charge monolayer has a mixed charge located both at the terminus of at least a subset of the molecules and along an interior chain of at least a subset of the molecules.

In another embodiment, the protective layer has an electrical capacitance that is a value selected from the group consisting of less than 200 μF/cm², less than 20 μF/cm² , less than 10 μF/cm², less than 5 μF/cm², less than 3 μF/cm², and less than 1 μF/cm². In one embodiment, the capacitance is limited by a non-monolayer blocking layer onto which the monolayer is attached. In another embodiment, the non-monolayer blocking layer comprises a material selected from the group consisting of semiconductor, metal oxide, nitride, carbide, and mixtures thereof. In one embodiment, the sample fluid is a biofluid selected from the group consisting of interstitial fluid, serum, and blood. In another embodiment, the device also includes an electrode and the electrode carries the protective layer.

In one embodiment, the sensor has an initial redox tag current when placed into the sample fluid and the redox tag current decreases by an amount selected from the group consisting of <5%, <10%, and <30% during the first six hours of operation at a temperature of at least 30 degrees Celsius. In another embodiment, the sensor has sensor response, and the sensor response when placed into the sample fluid decreases by an amount selected from the group consisting of <5%, <10%, and <30% during at least 3 days of operation at a temperature of at least 30 degrees Celsius. In one embodiment, the sensor has sensor response, and the sensor response when placed into the sample fluid decreases by an amount selected from the group consisting of <5%, <10%, and <30% during at least 7 days of operation at a temperature of at least 30 degrees Celsius.

In another embodiment, the sensor has an oxygen reduction current measurable at −0.5V, and the oxygen reduction current increases by an amount selected from the group consisting of <10%, <30%, and <100% compared to the magnitude of background current for at least 3 days of operation. In one embodiment, the sensor has an oxygen reduction current measurable at −0.5V, and the oxygen reduction current increases by an amount selected from the group consisting of <10%, <30%, and <100% compared to the magnitude of background current for at least 7 days of operation. In another embodiment, the mixed charge monolayer has a net charge density and the net charge density is less than a percentage selected from the group consisting of 50%, 25%, 5%, and 1% of an equivalent net charge of 4 electron charge per nm². In one embodiment, the mixed charge monolayer has a net charge and the net charge is negative.

In another embodiment, the mixed charge monolayer has a net charge and the net charge is positive, and further, a voltage is used to operate the sensor and the voltage is sufficiently negative such that at the boundary between the sample fluid and the monolayer the net potential is negative. In one embodiment, most of the first charge and most of the second charge are separated by a distance selected from the group consisting of 0.5, 1.0, and 1.5 nm. In another embodiment, most of the first charge and most of the second charge are separated by a number of methyl groups selected from the group consisting of 5, 10, and 15. In one embodiment, most of the first charge and most of the second charge are separated by a number of phenyl groups selected from the group consisting of 1, 2, and 3.

In another embodiment, the first charge has a first distance from the electrode while the positive charge has a second distance from the electrode, and the first distance is greater than the second distance such that the boundary between the monolayer and the sample fluid has a net negative charge. In one embodiment, the mixed charge monolayer has a density selected from the group consisting of at least 0.5, 1, 2, 3, 4 and 5 molecules/nm². In another embodiment, the device also includes a background current, and the background current increases by less than a percentage selected from the group consisting of 5%, 10%, 20% and 40% over at least 3 days. In one embodiment, the device also includes a background current, and the background current increases by less than a percentage selected from the group consisting of 5%, 10%, 20% or 40% over at least 7 days. In another embodiment, the mixed charge monolayer molecules include a long hydrophobic alkyl chain length selected from the group consisting of 6 methyl groups and 8 methyl groups in length.

In one embodiment, the mixed charge monolayer molecules are attached to the device using a chemistry selected from the group consisting of thiols, silanes, phosphonic acids, trichlorosilanes, trimethoxysilanes, triethoxysilanes, phosphates, alkenes, and alkynes. In another embodiment, the tag is a redox tag, and the device has an electron transfer rate, wherein the electron transfer rate changes by less than a percentage selected from the group consisting of 50%, 20%, 10%, and 5% over at least 3 days. In one embodiment, the tag is a redox tag, and the device has an electron transfer rate, wherein the electron transfer rate changes by less than a percentage selected from the group consisting of 50%, 20%, 10%, and 5% over at least 7 days. In another embodiment, the boundary between the protective layer and the sample fluid is within a value selected from the group consisting of +/−5 mV, +/−10 mV, +/−20 mV, +/−40 mV, and +/−80 mV of zero volts surface potential.

In one embodiment, the boundary between the protective layer and the sample fluid is positive in net charge without any applied voltage to the electrode, and a negative voltage is used during operation of the electrode. In another embodiment, the boundary between the protective layer and the sample fluid is negative in net charge without any applied voltage to the electrode, and a positive voltage is used during operation of the electrode. In one embodiment, the device also includes a plurality of molecular brush molecules interspersed between aptamers. In another embodiment, the boundary layer and the mixed charge monolayer have a Debye length, and further, wherein the molecular brushes have a net charge near the aptamers and which extend the Debye length from the boundary layer with the mixed charge monolayer by at least 2× in length.

In one embodiment, the sample fluid is interstitial fluid inside a human body. In another embodiment, the device also includes a first surface area in between the sample fluid and the sensor, and wherein there is <30% area coverage of foreign-body-response induced cells on the first surface area for a period of time selected from the group consisting of 3 days, 7 days, and 14 days. In one embodiment, the device also includes a first surface area in between the sample fluid and the sensor, and wherein there is <10% area coverage of foreign-body-response induced cells on the first surface area for a period of time selected from the group consisting of 3 days, 7 days, and 14 days. In another embodiment, the at least one tag is a redox tag and the parameter is redox current. In one embodiment, the parameter is electrical impedance. In another embodiment, the parameter is intensity of light.

In one embodiment, the device also includes a binding affinity between the analyte and the plurality of aptamers, and wherein said binding affinity is within at least one of +/−10, 50, 100, 200, 300, 500, and 1000% of the analyte binding affinity as tested for the same aptamers in the same manner except for using mercaptohexanol or mercaptooctanol in place of the mixed charge monolayer.

In another embodiment, the present invention involves a method of sensing at least one analyte in a sample fluid via measurement of the analyte. The method involves exposing the sample fluid to a sensor as described above and using data regarding changes in one or more aptamer parameters to measure the analyte. A voltage is applied to the electrode such that potential at the boundary between monolayer and the sample fluid has a net negative potential such that most foulants and aptamers are slightly repelled from the surface.

### BRIEF DESCRIPTION OF THE DRAWINGS

The objects and advantages of the disclosed invention will be further appreciated in light of the following detailed descriptions and drawings in which:

FIG. 1A is a schematic of one embodiment of a conventional prior art sensor device.

FIG. 1B is a schematic of another embodiment of a conventional prior art sensor device.

FIG. 2 is a schematic of one embodiment of a device in accordance with principles of the present invention.

FIG. 3 is a schematic of another embodiment of a device in accordance with principles of the present invention.

FIG. 4 is a schematic of yet another embodiment of a device in accordance with principles of the present invention.

FIG. 5A is a schematic of a prior art device.

FIG. 5B is a schematic of yet another embodiment of a device in accordance with principles of the present invention.

FIG. 5C is a schematic of yet another embodiment of a device in accordance with principles of the present invention.

FIG. 6A is a schematic of electrical measurement techniques using square wave voltammetry.

FIG. 6B is a graph showing “forward,” “backward” and “net” voltammograms.

FIG. 6C is a graph showing frequencies where the sensor response is ‘ON’ (increasing redox current) or alternately ‘OFF’ (decreasing redox current) as analyte binds to the aptamer.

FIG. 7A is a schematic of yet another embodiment of a device in accordance with principles of the present invention.

FIG. 7B is a schematic of yet another embodiment of a device in accordance with principles of the present invention.

FIG. 8 is a graph showing titration results as a plot of sensor response vs. total cortisol concentration.

### DEFINITIONS

As used herein, “continuous sensing” with a “continuous sensor” means a sensor that changes in response to changing concentration of at least one solute in a solution such as an analyte. Similarly, as used herein, “continuous monitoring” means the capability of a device to provide multiple measurements of an analyte over time.

As used herein, the term “about,” when referring to a value or to an amount of mass, weight, time, volume, pH, size, concentration or percentage is meant to encompass variations of ±20% in some embodiments, ±10% in some embodiments, ±5% in some embodiments, ±1% in some embodiments, ±0.5% in some embodiments, and ±0.1% in some embodiments from the specified amount, as such variations are appropriate to perform the disclosed method.

As used herein, the term “electrode” means any material that is electrically conductive such as gold, platinum, nickel, silicon, conductive liquid infused materials such as ionic liquids, PEDOT: PSS, conductive oxides, carbon, boron-doped diamond, nanotubes or nanowire meshes, or other suitable electrically conducting materials.

As used herein, the term “protective layer” means a monolayer protective layer or a non-monolayer protective layer, or a combination of both.

As used herein, the term “protective monolayer” means a homogeneous or heterogeneous layer of material or of one or more types of molecules which enables at least one of: reduced electrochemical background current and/or current due to electrochemical interference; reduced fouling in a sample fluid; and which may promote proper freedom of movement for the aptamer which is required for creating a measurable response to analyte concentration.

As used herein, the term “non-monolayer protective layer” means a homogeneous or heterogeneous layer of material or of one or more types of molecules on an electrode which do not represent a monolayer configuration, and which enables at least one of: reduced electrochemical background current and/or current due to electrochemical interference; reduced fouling in a sample fluid; and which may promote proper freedom of movement for the aptamer which is required for creating a measurable response to analyte concentration. For example, a metal or semiconductor oxide can be a non-monolayer protective layer, or a thin polymer film may be a non-monolayer protective layer, because they are comprised of multiple layers of atoms or molecules. A single atomic monolayer of SiO₂ for example would be a monolayer, whereas 3 nm of SiO₂ is a non-monolayer. A non-monolayer protective layer may also be for example a nitride, such as silicon nitride, or may be other suitable materials that serve a similar function including carbides and other suitable materials.

As used herein, the term “mixed charge monolayer” may be a protective monolayer and means a monolayer of at least partially vertically oriented molecules on a surface, comprising at least a first plurality of molecules with a first polarity of charge at or near their terminus facing the sample fluid, and at least a second plurality of molecules with a second polarity of charge at or near their terminus facing the sampling fluid, where the first polarity and second polarity are oppositely charged. A mixed charge monolayer may also include a monolayer that includes non-charged molecules such as mercaptohexanol or mercaptooctanol or other non-charged molecules so long as at least one percent (1%) of the molecules in the monolayer are mixed charges because mixed charges even if not at 100% of the molecules in the monolayer will increase monolayer surface hydration and the ability to prevent fouling of the monolayer surface, and/or can be used to modify the net surface potential which can impact the aptamers binding affinity to the analyte.

As used herein, the term “aptamer” means a molecule that undergoes a conformation or binding change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers and other affinity-based probes. Modifications may include substituting unnatural nucleic acid bases for natural bases within the aptamer sequence, replacing natural sequences with unnatural sequences, or other suitable modifications that improve sensor function, but which behave analogous to traditional aptamers. Two or more aptamers bound together can also be referred to as an aptamer (i.e., not separated in solution). Aptamers can have molecular weights of at least 1 kDa, 10 kDa, or 100 kDa.

As used herein, the term “tag” is a molecule carried on an aptamer that has a measurable response as analyte binds to the aptamer, such as a redox tag, or for example a fluorescent tag or quencher tag like that used in molecular beacons, or some other suitable tag that is measurable. For a redox tag the aptamers are typically carried on or near an electrode, and if fluorescent tag is utilized often the aptamers are carried on or near an optical waveguide.

As used herein, the term “redox tag” or “redox molecule” means any species such as small or large molecules with a redox active portion that when brought adjacent to an electrode can reversibly transfer at least one electron with the electrode. Redox tag or molecule examples include methylene blue, ferrocene, quinones, or other suitable species that satisfy the definition of a redox tag or molecule. In some cases, a redox tag or molecule is referred to as a redox mediator. Redox tags or molecules may also exchange electrons or change in behavior when brought into proximity with other redox tags or molecules. Exogenous redox molecules are those added to a device, e.g., they are not endogeneous and provided by the sample fluid to be tested.

As used herein, the term “change in electron transfer” means a redox molecule whose electron transfer with an electrode has changed in a measurable manner. This change in electron transfer can, for example, originate from availability of electrons to transfer, distance from an electrode, diffusion rate to or from an electrode, a shift or increase or decrease in electrochemical activity of the redox molecule, or any other embodiment as taught herein that results in a measurable change in electron transfer between the redox molecule and the electrode.

As used herein, the term “sensing monolayer” means at least a plurality of aptamers on a working electrode, which may also include a plurality of molecules or mixtures of molecules that form a non-monolayer protective layer or monolayer protective layer.

As used herein, the term “analyte” means any solute in a solution or fluid or sample fluid which can be measured using a sensor. Analytes can be small molecules, proteins, peptides, electrolytes, acids, bases, antibodies, molecules with small molecules bound to them, DNA, RNA, drugs, chemicals, pollutants, or other solutes in a solution or fluid.

As used herein, the term “sample fluid” is the fluid containing the analyte.

As used herein, a “device” comprises at least one sensor based on at least one aptamer, and at least one sample solution. Devices can sense multiple samples and be in multiple configurations such as a device to measure a pin-prick of blood, or a microneedle or in-dwelling sensor needle to measure interstitial fluid, or a device to measure saliva, tears, sweat, or urine sensor, or a device to measure water pollutants or food processing solutes, or other devices which measure at least one analyte found in a sample solution.

As used herein: “redox tag current” is the amplitude of the faradaic redox tag peak current (such as that collected in voltammogram) minus the background current amplitude outside the redox peak in a given voltammetric scan; “normalized redox-tag current” is the redox-tag current normalized to the first measurement taken; “background current” is the current that would be measured if the aptamer molecules were not tagged with a redox reporter including, for example, capacitive currents and competing redox processes such as oxygen reduction; “adjusted current” is the combined redox tag current and background current of a square-wave voltammogram adjusted such that the minimum current is set to 0 A in the presentation of the voltammogram such that voltammograms can be plotted side by side and compared with greater ease; “sensor response” is the change in redox tag current due to binding of the target analyte to the aptamer, also known as signal gain, which can either increase or decrease based on the aptamer and the voltametric time scale (such as operating frequency for square wave voltammetry). The above terms can be used for representation or interpretation in other types of sensor scanning techniques such as chronoamperometry and should not be narrowly interpreted in the specifically to only represent scanning methods such as square wave voltammetry.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a reference or counter electrode, a battery, antenna, adhesive), and for purposes of brevity and focus on inventive aspects, such components may not be explicitly shown in the diagrams or described in the embodiments of the disclosed invention. All ranges of parameters disclosed herein include the endpoints of the ranges.

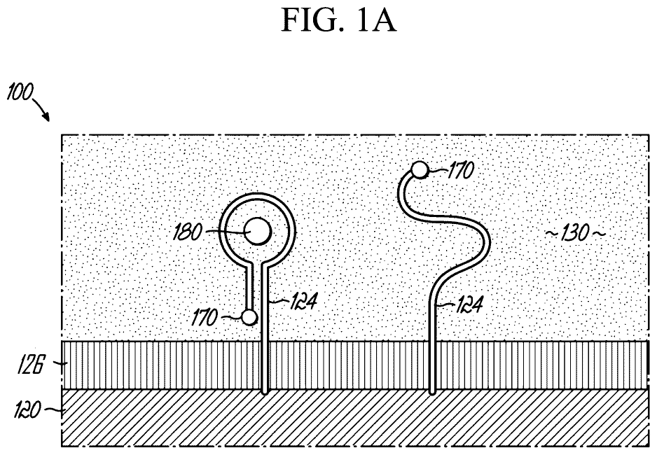

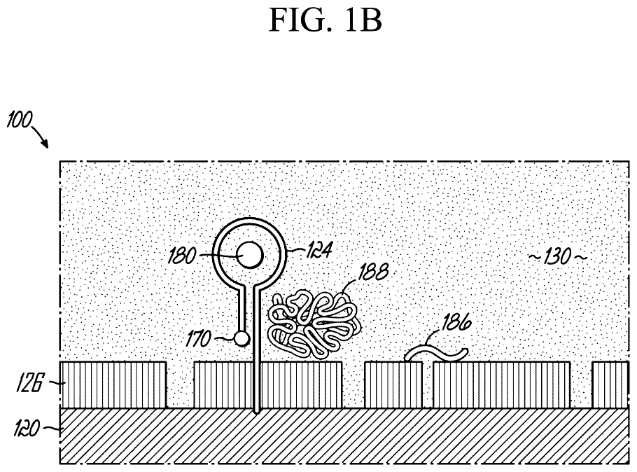

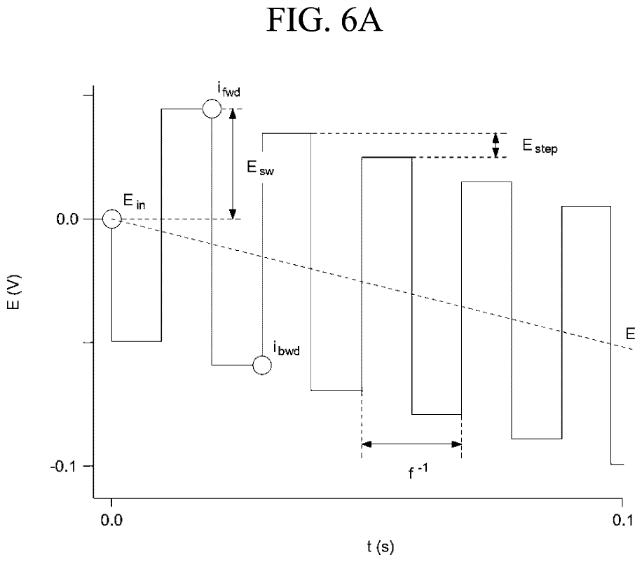

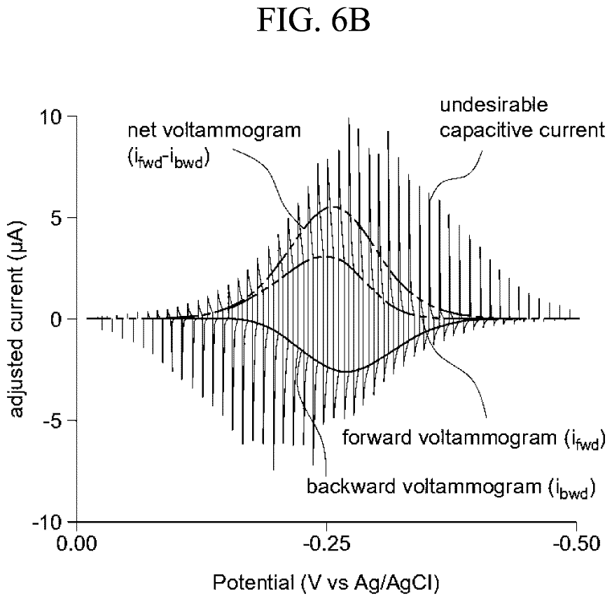

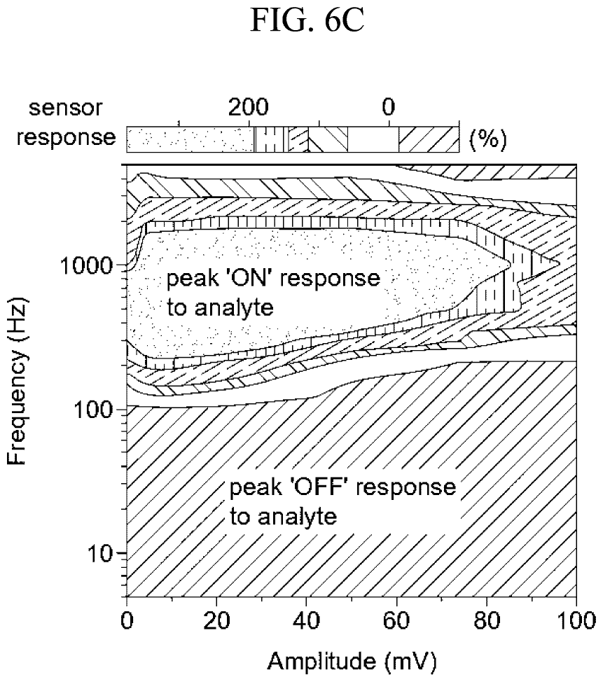

With reference to FIGS. 1A and 1B, a conventional prior art sensor device 100 as placed initially in a sample fluid 130, such as interstitial fluid, is shown. The device 100 includes at least one working electrode 120 such as gold, carbon, or other suitable electrode material; at least one protective layer that is a monolayer protective layer 126 such as a plurality of molecules such as mercaptohexanol that are thiol bonded to the electrode; at least one aptamer 124 that is responsive to binding to an analyte 180; and a redox tag 170, such as methylene blue, associated with at least one aptamer, such as by being bound thereto. In the generic example taught for FIGS. 1A and 1B, the aptamer 124 is a simple stem loop (hairpin) aptamer where analyte 180 binding causes the stem loop to form and the redox tag current measured from the redox tag 170 to increase, as measured using square wave voltammetry (FIGS. 6A, 6B), chronoamperometry, or other suitable technique. In absence of analyte 180 binding to the aptamer 124 the stem loop conformation does not form and the redox current thus does not increase. Alternately, analyte 180 binding to different example aptamer can cause a decrease in redox tag current. Thus, with aptamer sensors changes in a measurement of electrical redox tag current can be used as a signal to interpret changes in concentration of the analyte 180.

With further reference to FIG. 1B, a challenge with aptamer sensors is that when placed into initial operation the sample fluid 130, over a period of minutes to hours to days depending on the protective layer 126, the signal (e.g., redox tag current) decreases by 30%, 50%, or even more, due to effects such as fouling by small molecules 186, proteins 188, or other solutes in the sample fluid 130, but also due to desorption of the sensing monolayer, including aptamer 124 and/or protective layer 126.

As can be seen, currently available sensor devices include several drawbacks and limitations, particularly resulting from the protective layers found on such current devices. Various aspects of the present invention, however, resolve such drawbacks and limitations (including the amount of initial or longer-term desorption, instability, and/or fouling of the sensing monolayer). In that regard, one aspect of the present invention is directed to a device for detecting the presence of, or measuring the concentration or amount of, at least one analyte in a sample fluid. Specifically, the present invention reduces or resolves fouling while providing at least one of: maintaining fast electron transfer rates; enabling a strong magnitude of electron transfer current; and enabling optimal binding affinity for the analyte to the aptamer and resulting range of detection for analyte.

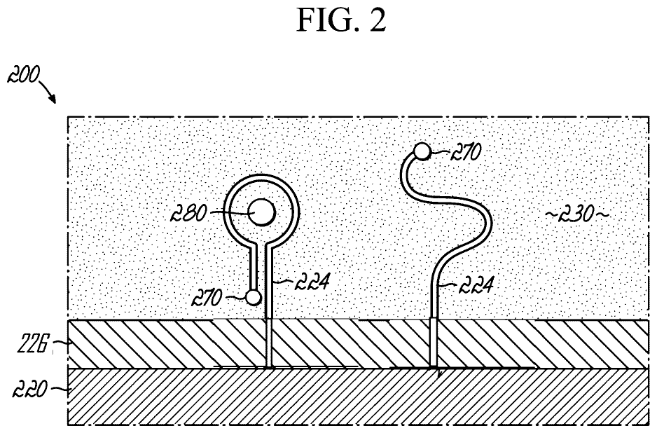

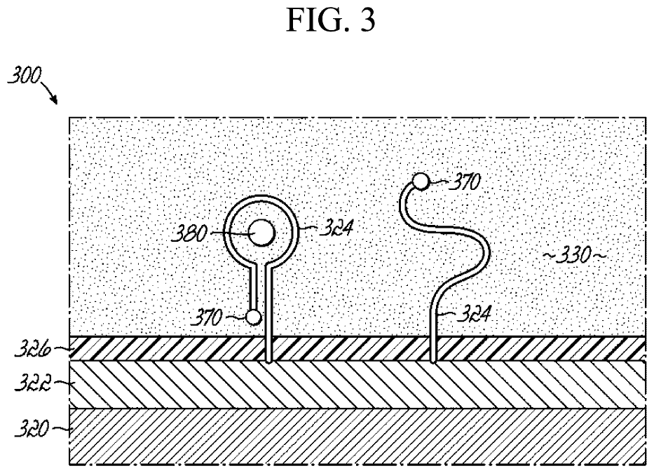

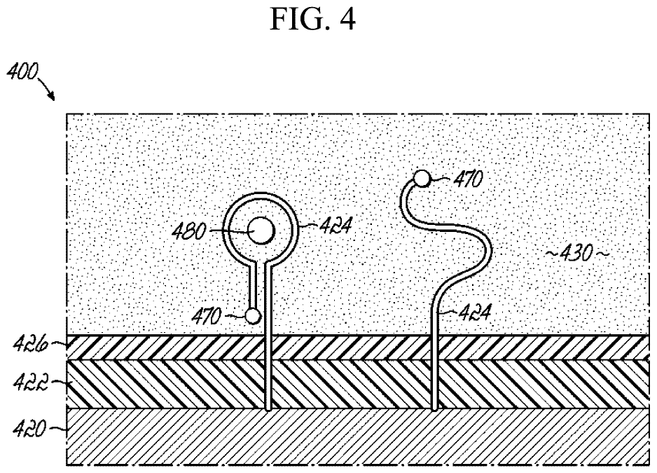

With reference to FIGS. 2, 3, and 4, several embodiments showing device structures for the present invention are first presented, where like numerals refer to like features. Devices include electrodes 220, 320, and 420, which could be metals such as gold or platinum, semiconductors such as silicon, conductive oxides such as In₂O₃:SnO₂, carbon such as glassy carbon or diamond like carbon, or other suitable electrodes. Electrodes such as glassy-carbon or diamond like carbon may be preferred as at an applied voltage of nearly −0.3V they suppress oxygen reduction current which is a form of background current by at least >3× compared to an electrode such as gold. Devices include at least one protective layer, that can be comprised at least in part of monolayers such as 226, 326, or 426. Protective layers may at least be comprised in part of a non-monolayer layer 322, 422 such as 1-2 nm of SiO₂, Al₂O₃, or other suitable material which is adequately porous to redox electron transfer. Devices may have aptamers attached to electrodes 220,420 or to non-monolayer layer 322. Attachment chemistries for aptamers and monolayers may include thiol, silanes, phosphonic acid, diazonium salts, or other suitable chemistries optimal for each electrode type as understood by those skilled in the art of monolayer chemistry. Monolayers may be formed of molecules consisting of chains of methyl groups like in aklthiolates, or phenyl groups in organosilanes, or other suitable chains.

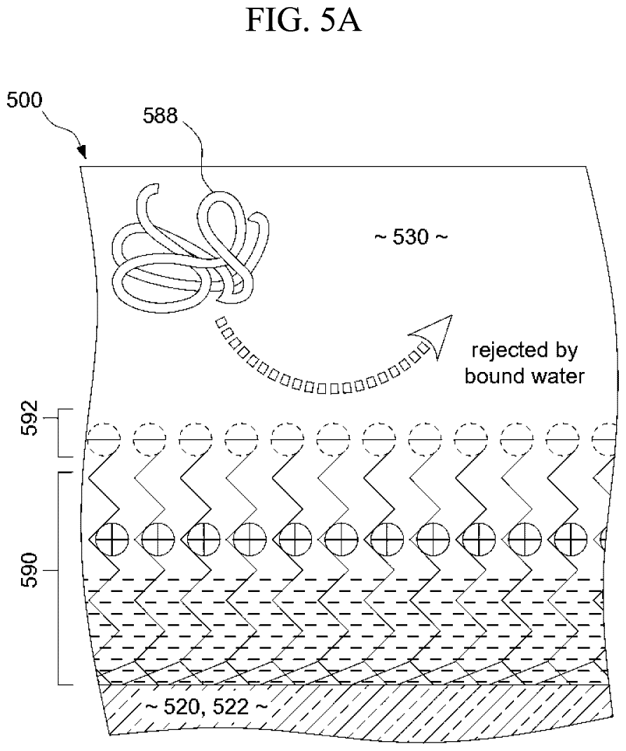

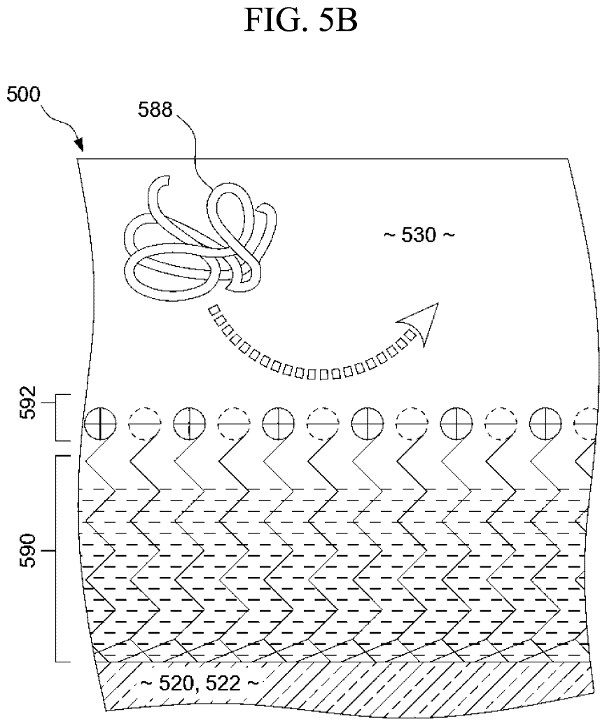

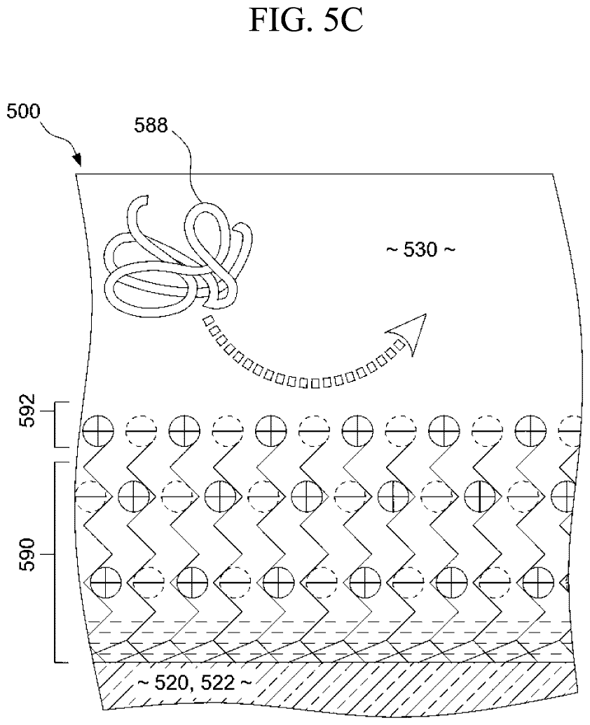

With reference to FIG. 5, prior art is illustrated in FIG. 5A and embodiments of the present invention are taught in FIG. 5B. One embodiment of the present invention shown in FIG. 5B uses a protective layer to significantly reduce fouling for electrochemical aptamer sensors by introducing mixed charges into the monolayer which spans distances 590 and 592, and preferably do so with a monolayer that is also stable with less desorption of prior art such as mercaptohexaonol monolayers as illustrated in FIG. 1B. The prior art in FIG. 5A is an example of a zwitterionic monolayer such as a phosphatidylcholine terminated monolayer (containing both + and − charge for each molecule in the monolayer at the pH of the sample fluid) and which can be attached to a substrate using thiol or other suitable chemistry. The prior art of FIG. 5A does provide two useful anti-fouling features, the zwitterionic charges promote binding of water in the monolayer (partially extending into depth 590) which acts as partial shield against fouling by repelling foulants 588, and the surface charge is negative in region 592 which is useful as most foulants such as albumin have a net negative charge. The prior art in FIG. 5A is able to reduce fouling but can present several potential disadvantages, including but not limited to; poor monolayer stability and resulting monolayer desorption; weaker electron transfer through the monolayer; electric field or steric effects on a negatively charged aptamer (not shown) such as negative surface charge as shown in FIG. 5A which repel and weaken binding affinity of an aptamer to its analyte; electric field or steric effects on the charged aptamer (not shown) such as positive surface charge which can attract a negatively charged aptamer to the surface and reduce its ability to bind with an analyte or to have a significant change in electron transfer due to binding; electric field or steric effects on a charged analyte, such as a negative surface charge as shown in FIG. 5A which can repel a negatively charged analyte to be measured and undesirably shift the binding affinity between aptamer and analyte; poorer packing density between adjacent molecules in the protective monolayer; or other potential disadvantages even if not specifically mentioned here.

With reference to FIG. 5B, mixed charges in region 592 locally create stronger dipoles as the oppositely charged elements of the dipole are brought more closely together near the monolayer boundary with the sample fluid 592, and therefore bind more water, reducing fouling. Although in FIG. 5B the charges are presented as highly periodic (alternating) they need not be so in practice. The present invention may further include zwitterionic charge in region 590 (not shown in FIG. 5B). Even more bound water is possible as illustrated in the embodiment of FIG. 5C where mixed charges are found at the boundary between monolayer and sample fluid (terminus facing the sample fluid 520) in region 592 and inside the monolayer region 590 (referred to herein as internal charge, and if mixed then referred to as internal mixed charge). As a result, if a foulant were to penetrate region 592, it would also have to penetrate region 590 and its bound water in FIG. 5C, which can be much more difficult to foul than the monolayer of FIG. 5B. The mixed charge monolayer chemistries spanning regions such as region 592 and/or 590 may also be attached to non-monolayer surfaces such as 322 and 422. With further references to embodiments of the present invention, in one embodiment, the monolayer is composed of zwitterions where one molecule is terminated in a phosphorylcholine group and the other molecule is terminated in a sulfobetaine group. This organization of molecules provides a zwitterionic monolayer comprising both a positively charged group on one molecule closer to the fluid interface and a negatively charged group on the other which is closer to the fluid interface with the opposite charge on each zwitterionic molecule being deeper into the monolayer. In another embodiment of the monolayer, each molecule is individually and oppositely charged, where one molecule is terminated in a positively charged group such as trimethylammonium and the other is terminated in a negatively charged group such as carboxylate.

As embodiments of the present invention are presented, additional explanation of operation of aptamer sensors is provided with respect to one of several possible measurement techniques called square wave voltammetry. FIG. 6A has a square waveform of frequency f and voltage amplitude E_{SW}. This is superimposed on a staircase that ramps the mean voltage (the mean potential per pulse pair, E) by a voltage step size per cycle, Estep. In FIG. 6B in response to this varying voltage, the system produces an oscillating faradic current. This current is then deconvoluted into “forward” and “backward” voltammograms by extracting the current at specific times after the capacitive background current has at least partially diminished (ifwd and ibwd). The difference between these two voltammograms then returns a “net” voltammogram that is more sensitive because the effect of capacitive charging currents are largely removed (because they are largely equal in magnitude regardless of polarity). As shown in FIG. 6C sensors can be designed such that the sensors have optimal frequencies where the sensor response is ‘ON’ (increasing redox current) or alternately ‘OFF’ (decreasing redox current) as the analyte binds to the aptamer. These frequencies are important because they not only represent maximum sensor response, but their ratios can be used to correct for sensor drift and enable ‘calibration free’operation so long as electron-transfer rates are stable.

With further reference to embodiments of the present invention, the protective layers comprised at least in part of elements 226, 322, 326, 422, 426 may have a mixed charge near the sample fluid 230, 330, 430, and should cumulatively not have too high of a total electrical capacitance between the sample fluid 230, 330, 430 and the electrode 220, 320, 420. Otherwise, techniques such as square-wave voltammetry cannot be utilized or will have increased electrical background current vs. measured redox tag current. For example, an alkythiolate monolayer protective layer with a dielectric constant of ~2.6 and ~0.7 nm or 1.4 nm thickness can have an electrical capacitance C=e*eo*A/t of C=2.6*8.854E−14 F/cm/7E−8 cm=3.3 μF/cm² or C=e*eo*A/t of C=2.6*8.854E−14 F/cm/ 1.4E−7 cm=1.6 μF/cm² The electrical double layer capacitance in most fluids (such as fluids 230, 330, 430) is so large that it is negligible in this calculation (due to thinness of the double layer in high salt conditions such as biofluids and the very high dielectric constant of water). This capacitance allows for square-wave voltammetry to be reliably performed generally up to ~1 kHz for methylene blue and up to ~10 kHz for redox tags such as Os(II/III). Therefore, the total electrical capacitance of the protective layer at measurement frequencies used for the device is at least one of less than 200 μF/cm² , less than 20 μF/cm², less than 10 μF/cm², less than 5 μF/cm² , or less than 3 μF/cm² or less than 1 μF/cm². For example, 200 μF/cm² could limit robust square wave voltammetry measurement frequency to ~10 Hz maximum and/or amperometrically or chronoamperometrically measured signals to those with slow electron transfer rates and/or weak signals. SiO₂ is therefore one preferred material for layers 322, 422 as it may dominantly reduce the capacitance between sample fluid and electrode, and the SiO₂ may be doped with carbon to lower its dielectric constant (capacitance) from ~4 to ~2.6-2.8, referred to herein as SiOC which may be ideal for enabling <1 μF/cm² capacitance. At equal capacitance a SiO₂ film that is 2 nm thick is equivalent to 3 nm of SiOC. The thickness and packing density of the mixed charge monolayer may also be designed to reduce the capacitance as well.

With further reference to embodiments for the protective layer of the present invention, the antifouling performance with direct exposure to human interstitial fluid or human serum at 33 ° C. as a testable surrogate, can be characterized in several ways including but not limited to the following. A first measure is raw redox current. Aptamer sensors typically when placed unprotected (no membrane protection) into interstitial fluid or serum, suffer an initial decrease in redox tag current of 50% and even 80% with rough gold electrodes, largely due to fouling of the surface which impedes redox electron transfer. The present invention with mixed charge monolayers may provide at least one of <5%, <10%, <30% loss in redox tag current during the first 6 hours of sensor operation in interstitial fluid or serum. The second measure is sensor response, which is change in redox tag current in response to changing analyte concentrations. Typically, without protection sensor response decreases due to foulants impeding freedom of movement for aptamers or due to other effects. The present invention with mixed charge monolayers may provide at least one of <5%, <10%, <30% loss in sensor response over at least 3 days of operation. For example, if a sensor when exposed to analyte at a first concentration has a change (increase or decrease) in redox tag current of 1 μA then after 3 days the change in redox tag current would be >0.8 μA when exposing the sensor again to that first concentration of analyte. As will be seen in the examples, a robust sensor response can be maintained for 7 days or more where day 4 data is nearly identical to the initial data and day 7 data only decreases by <5%, <10%, or <30%. A third measure is preventing an increase in oxygen reduction current measurable at −0.4 or −0.5 V and measurable in the sensor voltammograms during square wave voltammetry measurement of the sensor. The present invention with mixed charge monolayers may provide a measure of oxygen reduction current that increases by less than at least one of <10%, <30%, <100% compared to the magnitude of background current without significant oxygen reduction current for at least 7 days of operation. For a well-made sensor, the oxygen reduction current is near zero or <10% of the baseline current when the sensor is initially tested, which is a suitable non-limiting example of initial oxygen reduction current to use for later measurement of an increase in oxygen reduction current.

With further reference to embodiments for the protective layer of the present invention, the antifouling performance with direct exposure through a membrane that is at least a 100 kDa membrane and with human interstitial fluid or human serum at 33° C. as a testable surrogate, provides at least <10% reduction in redox tag current over the 1^{st} two hours of operation, and at least <30% loss in sensor response over at least 3 days of operation. The 100 kDa membrane is defined such that during the testing period the concentrations of large solutes and foulants >100 kDa (such as antibodies, or fibrogen) in fluid between the membrane and the sensor are held to less than 10% of the concentration of solutes >100 kDa in the sample fluid. For example, if a sensor when exposed to analyte has a change in redox tag current of 1 u A then after 3 days the change in redox tag current would be >0.8 μA. The present invention may further provide <30% loss in redox tag current over at least 7 days of operation. Membranes may generally be used with the present invention (not shown) and may be based on hydrogels, dialysis membranes, nanofiltration membranes, or other suitable materials. The mixed charge monolayers support such reduced fouling and the 100 kDa membrane, reduces large foulants that are so large that adsorption of just a few such foulants strongly impacts freedom of movement for aptamers adjacent to the foulant. Some examples of membranes which would be useful to protect such a mixed charge monolayer sensor include the 100 kDa Repligen Spectrum® Hollow Fiber Membranes or the 100 kDa Millipore NMWL Biomax polyethersulfone ultrafiltration membranes. Other membranes capable of reducing large protein access to the electrode surface could also be used for electrode protection.

With further reference to embodiments for the protective layer of the present invention, with direct exposure to human interstitial fluid or human serum at 33° C. as a testable surrogate, mixed charge monolayers may have a net charge that is at least one of less than 50%, 25%, 5%, or 1% of an equivalent a net charge density of 2 or of 4 electron charges per nm². Too strong of a net charge can both increase fouling by attracting oppositely charged foulants or can impart electrical force on the aptamer which can in some cases negatively affect sensor response. Aptamers and foulants typically, but not always, have negative charges. For example, if the mixed charge monolayer had a density of 4 monolayer molecules per nm² and 40% were positively charged and 60% were negatively charged at or near their terminus facing the sample fluid, then the monolayer would have a net charge per nm² that is negative and which has 0.2 charges*4 molecules/nm²=0.8 charges per nm². This would satisfy a mixed charge monolayers with a net charge that is at least one of less than 50% of an equivalent of a net charge of 1 charge per nm² (it is less than 1 charges per nm²). In another embodiment, a net charge is preferably negative such that most foulants and aptamers are slightly repelled from the surface. In one embodiment, a monolayer which satisfies a net negative charge contains molecules terminated in a sulfonate group such as 11-sulfanyl-1-undecanesulfonate plus molecules terminated in a trimethylammonium group such as N,N,N-trimethyl-11-sulfanyl-1-undecanaminium. For this monolayer, the sulfonate terminated molecule has a greater surface density than the trimethylammonium terminated molecule to provide the net negative charge. In another embodiment, a net charge is preferably positive, but a voltage is applied to the electrode such that potential at the boundary between monolayer and sample has a net negative potential such that most foulants and aptamers are slightly repelled from the surface. For example, a net charge could provide a surface potential at the sample fluid of +20 mV in the absence of applied voltage, and a square wave voltage scanned between −200 and −350 mV continuously scanned to create a net negative potential of at least −20 mV at the boundary between the mixed charge monolayer and the sample. The amount of applied voltage that appears at the boundary between the monolayer and sample depends on the porosity of the protective layer and charge accumulation within that protective layer. In one embodiment, a mixed charge monolayer is assembled for an aptamer sensor which contains one molecule terminated in a trimethylammonium group and another molecule terminated in a carboxylate group where the trimethylammonium terminated molecule has a greater density than the carboxylate group causing a positive surface potential at the sample fluid without an applied voltage. This greater density of positively charged molecules is obtained by modifying the assembly ratio of positive to negative molecule concentrations used during sensor preparation. Upon applying a negative voltage, the potential at the surface is closer to neutral than a sensor developed with mercaptohexanol but it still has an overall net negative surface potential to repel negatively charged foulants.

With further reference to embodiments for the protective layer of the present invention, with direct exposure to human interstitial fluid or human serum at 33° C. as a testable surrogate, mixed charge monolayers may have charges adequately separated either along the molecule chain that the charges are a part of or separated with respect to adjacent molecule chains. As a result of proper separation, acid-base reactions are avoided (and charge is not neutralized) and such that adequate distance exists to support inclusion of multiple bound water molecules. The charge separation may be defined multiple ways, and for example for charges at the boundary between the monolayer and the sample fluid, the oppositely charged charges are separated by, in alternative embodiments, 0.5, 1.0, or 1.5 nm, at least one of 5, 10, 15 methyl groups (carbons in an alkane chain), or at least one of 1,2, or 3 phenyl groups. In one embodiment, charges separated along the same chain is a choline group are attached to the molecule as the head group and a phosphate group and integrated into the alkane chain where the oppositely charged molecules are separated by 5 carbons to promote proper charge separation so that multiple water molecules are bound. Another embodiment of this concept is a sensor with a monolayer made up of molecules where one is terminated in a negative charge such as carboxylate and the other is terminated in a positive charge such as trimethylammonium. To promote proper spacing of these molecules, each molecule has a spacer side chain with a phenyl group attached which forces spacing of charges during monolayer assembly. In another embodiment, the negative charges have a first distance from the electrode while the positive charges have a second distance from the electrode, and the first distance is greater than the second distance such that near the surface negative potential and Debye screening occurs for negative charges and such that negatively charge species (such as aptamers or foulants such as albumin) are repelled from the monolayer surface. In one embodiment, a monolayer is created out of all the same molecule where a negatively charged group such as sulfonate is 1.5 nm from the electrode surface and is separated from a positively charged group such as trimethylammonium by 0.75 nm to provide proper Debye screening of the negative charge and allow the negatively charged species to repel common foulants.

With further reference to embodiments for the protective layer of the present invention, with direct exposure to human interstitial fluid or human serum at 33° C. as a testable surrogate, the mixed charge monolayer may have a density, in alternative embodiments, of at least 0.5, 1, 2, 3, 4, or 5 molecules/nm². Generally, a denser monolayer is more difficult for foulants to physically penetrate and foul, because as mixed charges are brought closer together the water in between them is more tightly bound (up to the limit of where acid-base reaction may occur for some charged molecules). In one embodiment, a mixed charge monolayer is made from molecules which are terminated in a negatively charged group such as phosphate for one and the other is terminated in a positively charged group such as trimethylammonium and the rest of the molecule is a sufficient length of carbons or other functional groups to promote a packing density after monolayer assembly of 1 molecule/nm².

With further reference to embodiments for the protective layer of the present invention, with direct exposure to human interstitial fluid or human serum at 33° C. as a testable surrogate, the mixed charge monolayer may maintain a background current that increases, in alternative embodiments, by less than 5%, 10%, 20% or 40% over at least 3 days or 7 days. Background current occurs outside the range of the redox potential peak, for example with a methylene blue redox tag vs. a Ag/AgCl reference the peak redox current is near −300 mV and background would be measured at for example −100 mV or −400 mV or −500 mV. Background current can increase by virtue of factors including increased porosity of the protective layer which can increase both electrical capacitance and charging current and/or oxygen reduction current, both of which can reduce the accuracy of the sensor. Therefore, while the protective layers of the present invention may comprise mixed charges in the monolayer, inclusion of such charges can destabilize the monolayer compared to a monolayer such as a pure alkythiolate monolayer such as mercaptooctanol. For a mercaptooctanol monolayer on a gold electrode, when properly assembled on the correct gold roughness and with proper electrical scanning the hydrophobic interactions between alkythiolate molecules in the monolayer can stabilize the monolayer for greater than 3 or even 7 days. Therefore, the present invention may utilize monolayers that likewise include a long hydrophobic alky chain length of at least 6, and more preferably at least 8 methyl groups (carbon units) to provide such hydrophobic interactions, and then such molecules can be terminated with mixed charges as taught herein. To enable even greater monolayer stability, electrodes or non-monolayer blocking layers may be comprised of non-gold materials such as carbon or oxides or other suitable materials such that monolayer molecules with mixed charges regardless of number of carbon units or hydrophobicity can be surface bound with greater stability than mono-thiol bonds on gold. Chemistries that are useful in the present invention include trimethoxysilanes, triethoxysilanes, phosphonic acids, trichlorosilanes, phosphates, alkenes, or alkynes. In one embodiment, a non-monolayer protection layer such as SiO₂ is deposited on a gold electrode to provide a surface for alternative chemistries. A combination of two phosphonic acids are used to create a mixed charge monolayer, where one is terminated in a primary amine and the other is terminated in a carboxylate group. In one embodiment, they are self-assembled on the SiO₂ substrate. At physiological conditions, the primary amine is positively charged and the carboxylate group is negatively charged, providing the mixed charges in the monolayer.

With further reference to embodiments for the protective layer of the present invention, with direct exposure to human interstitial fluid or human serum at 33° C. as a testable surrogate, the mixed charge monolayer may provide a redox tag current that is sufficiently strong for accurate sensor measurement, such as +/−10% or +/−20% sensor accuracy. If a protective layer is too thick or dense, it may suppress fouling and background current, but do so at the cost of amount of redox tag current. Therefore, the protective layer must stay permeable to electron transfer between the redox tag and the electrode. Furthermore, electron transfer rates should stay stable to reduce measurement inaccuracy and to allow two-frequency or continuous-square wave calibration free operation. Electron transfer rates should change by, in alternative embodiments, less than 50%, 20%, 10%, or 5% over at least 3 days or at least 7 days. In one embodiment, a mixed charge monolayer is assembled for an aptamer sensor out of a combination of molecules where one is terminated in a phosphorylcholine group and the other is terminated in a sulfobetaine group and the chain length of each molecule is long enough to promote favorable stability interactions between molecules, but also short enough as to not impede initial electron transfer rates. After continuous scanning over, in alternative embodiments, at least 3 days or at least 7 days, the sensor will remain stable due to the mixed charge monolayer so that the electron transfer rates change by less than 20%.

With further reference to embodiments for the protective layer of the present invention, with direct exposure to human interstitial fluid or human serum at 33° C. as a testable surrogate, the mixed charge monolayer may be made optimally compatible with the voltage scanning used for the aptamer sensor such that a strong positive or negative potential is not experienced at the boundary between the protective layer and the sample fluid. A strong positive or negative potential at the boundary between the protective layer and the sample fluid may aid fouling by allowing charged foulants or portions of foulants with strong dipoles to penetrate through the bound water at the boundary between the protective layer and the sample fluid. For example, a protective layer could be used that has net negative charge at the boundary between protective layer and sample, and the applied voltage during measurement continuously applied in a manner such that the net surface potential at the boundary between the protective layer and the sample is within at least one of +/−5 mV, +/−10 mV, +/−20 mV, +/−40 mV, or +/−80 mV of zero volts surface potential. For methylene blue which requires negative voltages for scanning the mixed charge monolayer would be positive in net charge. For Os(II/III) redox tags or ferrocene redox tags which require positive or near neutral voltages for scanning the mixed charge monolayer would be negative or near neutral, respectively, in net charge. Other combinations are possible, such as a redox tag operating near zero volts for redox tag current and therefore a mixed monolayer that is at or near zero net charge. The voltage scanning may also be incompatible with reducing fouling but kept short in duration to minimize resulting fouling. For example, consider a near neutral or neutral net charge monolayer and an Os(II/III) or ferrocene redox tag that is scanned with positive voltage that can attract many foulants to or into the mixed charge monolayer. Instead of using a full square wave voltametric scan over several hundred millivolts, which can take a full minute to complete, only potentials near the redox peak potential can be scanned. For example, at 300 Hz scanning (3.3 ms total for the negative and positively trending pulse in FIG. 6A) a total of 5 current samples can be taken for a total of only 16.5 ms of scanning. If a measurement is taken every 5 minutes in this manner, the voltage duty cycle is 16.5E−3/(5*60)=0.0055% which is a tiny duty cycle and tiny period over which scanning is performed and during which fouling could be increased. Furthermore, once the surface is fouled, applied voltages can reorganize the foulants (such as albumin) into alternate geometries that allow additional foulants to adhere and to then progressively foul the surface. Therefore, the present invention may further comprise a measurement circuit such as wearable and programmable potentiostat and may utilize partial voltammogram scanning which only captures a portion of the peak redox tag current. Therefore, the present invention may utilize scanning with a scanning duty cycle of at least one of less than 10%, 1%, or 0.1% during operation of the device.

With further reference to embodiments for the protective layer of the present invention, the mixed charge layer can strongly impact the freedom of movement and/or binding affinity of the aptamer with the analyte. Several non-limiting examples are now provided. For example, in some cases a strong sensor response is often possible with aptamers with mercaptohexanol protective monolayers because these aptamers have a net negative potential and the mercaptohexanol boundary with the sample fluid has a net negative potential that repels the aptamer and redox tag, and this repulsion and distancing of the redox tag is maintained until the aptamer binds the analyte and forms a stem loop configuration (e.g. like FIG. 1) which brings the redox tag closer to the electrode. Such a configuration results in a strong sensor response (change in redox tag current). If the protective monolayer was positively charged then aptamer and redox tag may more often or even always be closer to the electrode due to electrostatic attraction, resulting in a weaker sensor response. As another example, the electric field and Debye screening layer extends far enough out from a mercaptohexanol blocking layer such that it can inhibit analyte binding with the aptamer. Therefore, the charge of the aptamer and the mixed protective monolayer can in some cases be very important to sensor response and binding affinity of the aptamer. The present invention may therefore impart a net positive charge on the aptamer or impart a net positive charge near the redox tag on the aptamer via tagging the aptamer with positively charged species or chemical modification of the aptamer or, for example, incorporation of positively charged primary amines through flexible linkers. As a result, the binding affinity or sensor response of the aptamer can be optimized compared to a natural DNA aptamer with only negative charge due to the phosphate backbone of the aptamer. Numerous combinations of mixed charge protective layers and aptamer charges are possible, along with applied voltages to the electrode, to therefore optimize the performance of the sensor such as binding affinity or sensor response. For example, for protein sensing the aptamer can have at least +1, +2, +3 electron charges within 10 nucleotide units of the redox tag such that a mixed charge protective layer with a net negative potential at the boundary with the sample fluid will have a strong redox tag current (as the redox tag is electrostatically brought closer to the electrode). With protein binding the aptamer in most cases will have to conform its shape to the large protein which requires in most cases the aptamer to move the redox tag away from the electrode, resulting in a strong sensor response (albeit a decreasing redox tag current as the protein analyte concentration increases).

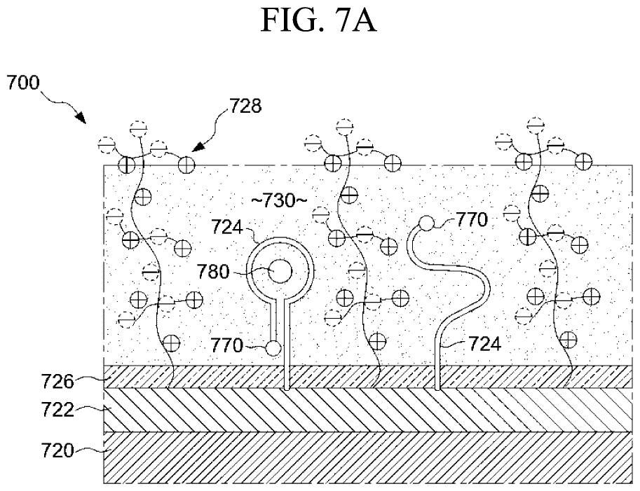

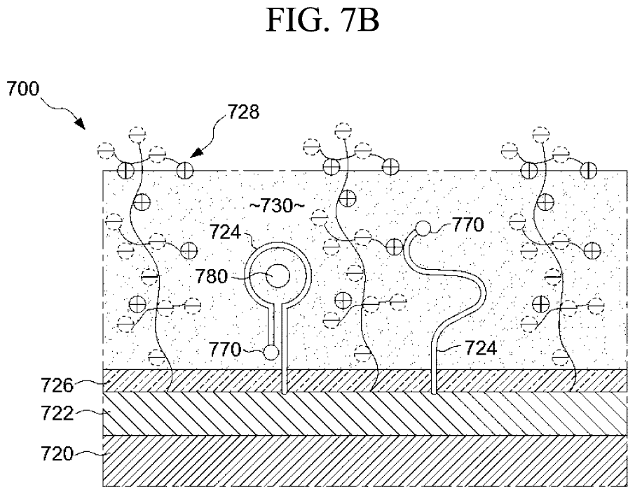

With further reference to embodiments for the protective layer of the present invention, in human interstitial fluid or human serum at 33° C. the Debye length is approximately 0.7 nm and is the distance over which the applied voltage and resulting electric potential in the sample fluid drops to 1/e of its potential at the surface (assuming the voltage abruptly meets the sample fluid with no voltage loss in between). As a result, the redox tag cannot be distanced too far from the potential drop near the electrode, and as a further result there is an upper limit on the thickness of the mixed charge monolayer for several reasons including: (1) if the mixed charge monolayer is highly electrically insulating and too thick then too much of the applied voltage will drop too much across the monolayer instead of the sample fluid (wherein the redox tag is located); (2) if the mixed charge monolayer is partially electrically conductive such that charges can penetrate it within the time scales of measurement (such as microseconds to milliseconds) then again the applied voltage will drop too much across the monolayer instead of the sample fluid (wherein the redox tag is located). This is unfortunate, because it therefore precludes use of long-chain length molecular brushes and other 1's of nm thick antifouling layers which have superior antifouling performance. However, in the present invention these antifouling chemistries may be incorporated along with the mixed charge monolayer at a density such that freedom of movement of the aptamer is not inhibited nor the redox tag further distanced from the electrode. Such densities generally are, but not limited to, at least one of <10¹³/cm², <10¹²/cm², <10¹¹/cm², or <10¹⁰/cm². As illustrated in FIG. 7A, these long-change molecular brushes such as polyethylene glycol (PEG), or polyacrylamides, may be >1 nm in length then may also contain mixed charges and repel very large size foulants without need for a protective membrane such as a 100 kDa membrane. Furthermore, as illustrated in FIG. 7B because Debye length extension can improve aptamer binding affinity and sensor response, the near-aptamer portions of such long-chain molecular brushes can be negatively charged to extend the Debye length, as for example the case of negative voltage redox tag such as methylene blue and/or a negative surface potential at the protective layer boundary with the sample fluid. Therefore, the present invention may further include a plurality of molecular brush molecules interspersed between aptamers. Therefore, the present invention may further include a plurality of molecular brush molecules interspersed between aptamers that have a net charge near the aptamers and which extend the Debye length from the boundary layer with the mixed charge monolayer by at least 2× in length.

With further reference to embodiments of the present invention a sensor may be constructed with a mixed charge monolayer anti-fouling capability that limits cell deposition and fibrogen growth on the sensor. Such an embodiment may further include molecular brushes as taught herein. Such an embodiment may include a zwitterionic cross-linked membrane film such as polybetaine suspended above the sensor to separate the sensor from biofluid, or which is deposited onto the sensor with a density that still permits freedom of movement for the aptamers and therefore proper sensor response. For wearable glucose monitors the lag time between blood and the glucose (>10 minutes) is limited by the foreign body response layer and tissue deposition on the sensor membrane which can be multiple layers of cells thick. If a tiny 180 Da such as glucose will find a tortuous path through this deposited tissue layer on the sensor, then analytes larger than >1 kDa and especially proteins will have much greater difficulty in reaching the sensor surface through the deposited tissue layer. The embodiments of the present invention can be tested against two use cases, insertion into skin or insertion into blood vessels or veins (both of which having been demonstrated in the art for short term use for aptamer sensors as an example). The embodiments of the present invention can be tested in these use case scenarios comprising a surface in between the sample fluid and sensor and the molecules and material carried on its surface, and wherein there is at least <10% or <30% area coverage of foreign-body-response induced cells for at least one of 3 days, 7 days, or 14 days. This cellular coverage is easily testable by retrieving the sensor after in-vivo use and inspecting the sensor surface via staining for cells or for fibrogen or other materials that would make up a deposited tissue layer.

With further reference to embodiments for the protective layer of the present invention, with direct exposure to human interstitial fluid or human serum at 33° C. as a testable surrogate, the present invention may also be deployed as an impedimetric aptamer sensor measured using electrical impedance measurement. While these sensors have struggled to perform in-vivo due to non-selective binding and fouling, the present invention remedies that limitation and permits their operation in in-vivo. Therefore, the present invention may include electrical impedance measurement of the sensors. The present invention may similarly enable biolayer interferometry, where the aptamers form a layer which changes in refractive index with binding of the analyte, and the aptamers are suspended at the end of an optical fiber, with a device structure similar to commercial biolayer interferometry devices but using advance antifouling capability as taught herein.

With further reference to embodiments for the protective layer of the present invention, with direct exposure to human interstitial fluid or human serum at 33° C. as a testable surrogate, the mixed charge monolayer may be made optimally compatible with molecular pendulum operation. Here, the analyte is a large protein and the aptamer when binding to the protein has a slower speed at which voltage on the electrode can repel or attract the negatively charged aptamer and protein. A slower time scale is measurable as a slower change in increase or decrease in redox tag current as the aptamer or aptamer and attached protein are respectively electrostatically attracted or repelled from the electrode.

With further reference to embodiments for the protective layer of the present invention, with direct exposure to human interstitial fluid or human serum at 33° C. as a testable surrogate, the mixed charge monolayer may allow aptamer sensors with analyte binding affinities with at least one of +/−10, 50, 100, 200, 300, 500, 1000% of the analyte binding affinity as tested for the same aptamers in the same manner except for using mercaptohexanol or mercaptooctanol in place of the mixed charge monolayer. For example, if a vancomycin aptamer sensor had a binding affinity with vancomycin of 50 μM when implemented with a mercaptohexanol monlayer, then the mixed charge monolayers of the present invention can be optimized in their surface charge (as taught herein) to maintain a binding affinity of at least 45 μM but not more than 55 M.

### EXAMPLES

### Example 1

Working electrodes were initially prepared as follows. Briefly, gold electrodes were polished for 1 minute with a rotating disk polisher in 0.3 μm and 0.05 μm alumina slurry. Next, they were sonicated for 5 minutes in ethanol rinsed with deionized water and sonicated for another 5 minutes in deionized water. After sonication, they were briefly rinsed again in deionized water. The electrodes were then electrochemically cleaned in 0.5 M NaOH by scanning with cyclic voltammetry between potentials of −1 to −1.6 V with respect to a standard Ag/AgCl reference electrode for 1400 cycles then in 0.5 M H₂SO₄ between potentials of 0 to +1.6 for 300 cycles. After electrochemical cleaning, the electrodes were electrochemically roughened with a NaOH roughening technique. Briefly, the electrodes were placed in 5 M NaOH a chronoamperometrically interrogated between the potentials of +1.32 and −4.48 with respect to a mercury oxide reference electrode. After electrochemical roughening, the electrodes were again electrochemically cleaned in 0.5 MH₂SO₄ and rinsed prior to aptamer deposition.

For aptamer deposition on the electrodes, 400 nM of cortisol aptamer was prepared in a HEPES buffer containing: 20 mM HEPES, 1 M NaCl, 10 mM MgCl₂, 5 mM KCI, and the pH was adjusted to 7.5 with HCI. 20 μL of aptamer was drop-casted on each electrode for 1 hour and placed in a humidity controlled light protected box. After the 1 hour, the mixed charge monolayer solution was prepared and each electrode was placed in 500 μL of mixed charge monolayer solution overnight. Four electrodes were placed in an equimolar solution of 5 mM 8-mercaptooctanoic acid and 5 mM 8-amino-1-octanethiol hydrochloride in 1×PBS. All chemicals were purchased from Sigma Aldrich.

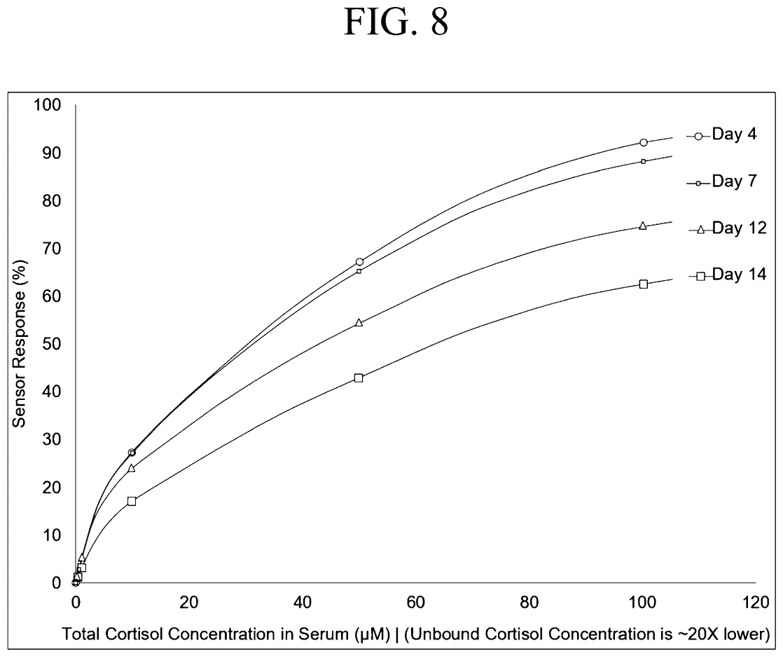

After incubation in the mixed charge monolayer, the electrodes were thoroughly rinsed in deionized water and setup to scan with square wave voltammetry in 1×PBS 100 times per electrode to remove any physically adsorbed species to the surface and better stabilize the electrode surface prior to testing. Then, the electrodes were placed in serum at 33 degrees Celsius and allowed to stabilize for 3 hours with intermittent square wave voltammetry (SWV) scanning every 15 minutes. After stabilization, electrodes were titrated with varying concentrations of cortisol to examine their current response across various frequencies. After titrating across the desired concentrations, the electrodes were placed in fresh serum and allowed to scan with SWV every 15 minutes for 4, 7, 12, and 14 days. At each of those days, the electrodes were titrated and returned to fresh serum to continue scanning for subsequent days. The resulting titration results are plotted in FIG. 8 as a plot of sensor response vs. total cortisol concentration. In serum, ~95% of cortisol is protein bound, so the concentration being measured by the sensor is approximately 5% of the total concentration added and labeled on the x-axis.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A device for continually sensing at least one analyte in a sample fluid via measurement of the analyte, comprising at least one sensor having a surface comprising:
a plurality of aptamers that bind to the analyte and wherein the plurality of aptamers carry at least one tag that changes in at least one parameter as analyte binds to the aptamers; and
a protective layer that protects the surface from fouling in between the aptamers, wherein the protective layer comprises a monolayer of molecules that form a boundary with the sample fluid;
wherein the monolayer of molecules is a mixed charge monolayer of at least a first molecule and a second molecule, the first molecule having a first charge that imparts a net negative charge at the boundary with the sample fluid, and the second molecule having a second charge that imparts a net positive charge near the boundary with the sample fluid.

2. The device of claim 1 wherein the first charge and the second charge have a magnitude of at least one electron charge.

3. The device of claim 1 wherein the monolayer of molecules comprises a repeating hydrophobic chain of molecules that is selected from the group consisting of two or more methyl groups, two or more phenyl groups, and combinations thereof.

4. The device of claim 1 wherein the mixed charge monolayer comprises one or more subsets of molecules, each subset of molecules having a terminus, and further, wherein the mixed charge monolayer has a mixed charge at the terminus of at least a subset of the molecules.

5. The device of claim 1 wherein the mixed charge monolayer has a mixed charge located along an interior chain of at least a subset of the molecules.

6. The device of claim 1 wherein the mixed charge monolayer has a mixed charge located both at the terminus of at least a subset of the molecules and along an interior chain of at least a subset of the molecules.

7. The device of claim 1 wherein the protective layer has an electrical capacitance that is a value selected from the group consisting of less than 200 μF/cm², less than 20 μF/cm², less than 10 μF/cm², less than 5 μF/cm², less than 3 μF/cm², and less than 1 μF/cm².

8. The device of claim 7 wherein the capacitance is limited by a non-monolayer blocking layer onto which the monolayer is attached.

9. The device of claim 8 wherein the non-monolayer blocking layer comprises a material selected from the group consisting of semiconductor, metal oxide, nitride, carbide, and mixtures thereof.

10. The device of claim 1 wherein the sample fluid is a biofluid selected from the group consisting of interstitial fluid, serum, and blood.

11. The device of claim 10 further comprising an electrode, wherein the electrode carries the protective layer.

12. The device of claim 11 wherein the sensor has an initial redox tag current when placed into the sample fluid and the redox tag current decreases by an amount selected from the group consisting of <5%, <10%, and <30% during the first six hours of operation at a temperature of at least 30 degrees Celsius.

13. The device of claim 10 wherein the sensor has sensor response, and the sensor response when placed into the sample fluid decreases by an amount selected from the group consisting of <5%, <10%, and <30% during at least 3 days of operation at a temperature of at least 30 degrees Celsius.

14. The device of claim 10 wherein the sensor has sensor response, and the sensor response when placed into the sample fluid decreases by an amount selected from the group consisting of <5%, <10%, and <30% during at least 7 days of operation at a temperature of at least 30 degrees Celsius.

15. £ The device of claim 11 wherein the sensor has an oxygen reduction current measurable at −0.5V, and the oxygen reduction current increases by an amount selected from the group consisting of <10%, <30%, and <100% compared to the magnitude of background current for at least 3 days of operation.

16. The device of claim 11 wherein the sensor has an oxygen reduction current measurable at −0.5V, and the oxygen reduction current increases by an amount selected from the group consisting of <10%, <30%, and <100% compared to the magnitude of background current for at least 7 days of operation.

17. The device of claim 10 wherein the mixed charge monolayer has a net charge density and the net charge density is less than a percentage selected from the group consisting of 50%, 25%, 5%, and 1% of an equivalent net charge of 4 electron charge per nm 2.

18. The device of claim 10 wherein the mixed charge monolayer has a net charge and the net charge is negative.

19. The device of claim 11 wherein the mixed charge monolayer has a net charge and the net charge is positive, and further, a voltage is used to operate the sensor and the voltage is sufficiently negative such that at the boundary between the sample fluid and the monolayer the net potential is negative.

20. The device of claim 1 wherein most of the first charge and most of the second charge are separated by a distance selected from the group consisting of 0.5, 1.0, and 1.5 nm.

21. The device of claim 1 wherein most of the first charge and most of the second charge are separated by a number of methyl groups selected from the group consisting of 5, 10, and 15.

22. The device of claim 1 wherein most of the first charge and most of the second charge are separated by a number of phenyl groups selected from the group consisting of 1, 2, and 3.

23. The device of claim 10 wherein the first charge has a first distance from the electrode while the positive charge has a second distance from the electrode, and the first distance is greater than the second distance such that the boundary between the monolayer and the sample fluid has a net negative charge.

24. The device of claim 1 wherein the mixed charge monolayer has a density selected from the group consisting of at least 0.5, 1, 2, 3, 4 and 5 molecules/nm².

25. The device of claim 11 further comprising a background current, and wherein the background current increases by less than a percentage selected from the group consisting of 5%, 10%, 20% and 40% over at least 3 days.

26. The device of claim 11 further comprising a background current, and wherein the background current increases by less than a percentage selected from the group consisting of 5%, 10%, 20% or 40% over at least 7 days.

27. The device of claim 1, wherein the mixed charge monolayer molecules include a long hydrophobic alkyl chain length selected from the group consisting of 6 methyl groups and 8 methyl groups in length.

28. The device of claim 1, wherein the mixed charge monolayer molecules are attached to the device using a chemistry selected from the group consisting of thiols, silanes, phosphonic acids, trichlorosilanes, trimethoxysilanes, triethoxysilanes, phosphates, alkenes, and alkynes.

29. The device of claim 11, wherein the tag is a redox tag, and the device has an electron transfer rate, wherein the electron transfer rate changes by less than a percentage selected from the group consisting of 50%, 20%, 10%, and 5% over at least 3 days.

30. The device of claim 11, wherein the tag is a redox tag, and the device has an electron transfer rate, wherein the electron transfer rate changes by less than a percentage selected from the group consisting of 50%, 20%, 10%, and 5% over at least 7 days.

31. The device of claim 11, wherein the boundary between the protective layer and the sample fluid is within a value selected from the group consisting of +/−5 mV, +/−10 mV, +/−20 mV, +/−40 mV, and +/−80 mV of zero volts surface potential.

32. The device of claim 31, wherein the boundary between the protective layer and the sample fluid is positive in net charge without any applied voltage to the electrode, and a negative voltage is used during operation of the electrode.

33. The device of claim 31, wherein the boundary between the protective layer and the sample fluid is negative in net charge without any applied voltage to the electrode, and a positive voltage is used during operation of the electrode.

34. The device of claim 1 further comprising a plurality of molecular brush molecules interspersed between aptamers.

35. The device of claim 34 wherein the boundary layer and the mixed charge monolayer have a Debye length, and further, wherein the molecular brushes have a net charge near the aptamers and which extend the Debye length from the boundary layer with the mixed charge monolayer by at least 2× in length.

36. The device of claim 10 where the sample fluid is interstitial fluid inside a human body.

37. The device of claim 36 further comprising a first surface area in between the sample fluid and the sensor, and wherein there is <30% area coverage of foreign-body-response induced cells on the first surface area for a period of time selected from the group consisting of 3 days, 7 days, and 14 days.

38. The device of claim 36 further comprising a first surface area in between the sample fluid and the sensor, and wherein there is <10% area coverage of foreign-body-response induced cells on the first surface area for a period of time selected from the group consisting of 3 days, 7 days, and 14 days.

39. The device of claim 1 wherein the at least one tag is a redox tag and the parameter is redox current.

40. The device of claim 1 wherein the parameter is electrical impedance.

41. The device of claim 1 wherein the parameter is intensity of light.

42. The device of claim 1 further comprising a binding affinity between the analyte and the plurality of aptamers, and wherein said binding affinity is within at least one of +/−10 , 50, 100, 200, 300, 500, and 1000% of the analyte binding affinity as tested for the same aptamers in the same manner except for using mercaptohexanol or mercaptooctanol in place of the mixed charge monolayer.

43. A method of sensing at least one analyte in a sample fluid via measurement of the analyte comprising exposing the sample fluid to the sensor of claim 1 and using data regarding changes in one or more aptamer parameters to measure the analyte, wherein a voltage is applied to the electrode such that potential at the boundary between monolayer and the sample fluid has a net negative potential such that most foulants and aptamers are slightly repelled from the surface.
